(19) United States
(12) Patent Application Publication
(10) Pub. No.: US 2025/0186705 A1
(43) Pub. Date: Jun. 12, 2025

(54) SUB-ASSEMBLY FOR A MEDICAMENT DELIVERY DEVICE

(71) Applicant: SHL Medical AG, Zug (CH)

(72) Inventors: Daniel Scott, Deerfield Beach, FL (US); Amit Singh, Deerfield Beach, FL (US)

(21) Appl. No.: 18/846,367

(22) PCT Filed: Feb. 28, 2023
(86) PCT No.: PCT/EP2023/054903

§ 371 Date: Sep. 12, 2024

Related U.S. Application Data
(60) Provisional application No. 63/319,474, filed on Mar. 14, 2022.

(30) Foreign Application Priority Data
May 13, 2022 (EP) 22173147.4

(51) Int. Cl. A61M 5/315

(52) U.S. Cl. CPC A61M 5/3157; A61M 5/31586

(57) ABSTRACT

The present disclosure provides a sub-assembly for a medicament delivery device, the sub-assembly extending along a longitudinal axis in a longitudinal direction from a proximal end to a distal end. The sub-assembly comprises a housing, and a plunger rod arranged at least partly inside the housing, the plunger rod being rotationally locked relative to the housing. The plunger rod comprises a flexible arm, the flexible arm comprising a first surface facing towards the proximal end. A component of the sub-assembly other than the plunger rod comprises a second surface facing towards the distal end, with the first surface and the second surface being arranged so that, when the plunger rod moves towards the proximal end relative to the component during medicament delivery device use, the first surface and the second surface engage to flex the flexible arm in a circumferential direction relative to the component and then disengage so that the flexible arm flexes back relative to the component.

### CROSS-REFERENCE TO RELATED APPLICATIONS

The present application is a U.S. National Phase Application pursuant to 35 U.S.C. § 371 of International Application No. PCT/EP2023/054903 filed Feb. 28, 2023, which claims priority to (i) U.S. Provisional Patent Application No. 63/319,474 filed Mar. 14, 2022, and (ii) European Patent Application No. 22173147.4 filed May 13, 2022. The entire disclosure contents of these applications are herewith incorporated by reference into the present application.

### TECHNICAL FIELD

The invention concerns sub-assemblies for medicament delivery devices, and particularly sub-assemblies with a housing and a plunger rod.

### BACKGROUND

Some medicament delivery devices provide feedback to their users during operation, such as with the U-bracket described in WO2011/123024. While such systems can provide effective feedback, the applicant has appreciated that an alternative feedback mechanism could usefully be provided, potentially providing various advantages when compared to existing feedback mechanisms.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

A sub-assembly for a medicament delivery device, the sub-assembly extending along a longitudinal axis in a longitudinal direction from a proximal end to a distal end, the sub-assembly comprising: a housing; and a plunger rod arranged at least partly inside the housing, the plunger rod being rotationally locked relative to the housing; wherein the plunger rod comprises a flexible arm, the flexible arm comprising a first surface facing towards the proximal end; wherein a component of the sub-assembly other than the plunger rod comprises a second surface facing towards the distal end, with the first surface and the second surface being arranged so that, when the plunger rod moves towards the proximal end relative to the component during medicament delivery device use, the first surface and the second surface engage to flex the flexible arm in a circumferential direction relative to the component and then disengage so that the flexible arm flexes back relative to the component (to create feedback). This can provide a feedback such as an audible, visible or tactile feedback to a medicament delivery device user.

Optionally, the component is the housing. Optionally, the housing comprises an outer housing and a rear cap, and the rear cap comprises the second surface. Optionally, the second surface is on a protrusion. Optionally, the protrusion extends towards the longitudinal axis.

Optionally, at least part of at least one of the first surface and the second surface is angled relative to a plane perpendicular to the longitudinal axis.

Optionally, the at least part of at least one of the first surface and the second surface is angled relative to a plane perpendicular to the longitudinal axis at an angle of between 10 and 85 degrees. Optionally, at least part of the first surface is angled relative to a plane perpendicular to the longitudinal axis.

Optionally, the sub-assembly comprises a feedback surface angled relative to a plane perpendicular to the longitudinal axis, wherein the feedback surface is spaced apart in a circumferential direction relative to the longitudinal axis from a distal end of the first surface.

Optionally, at least part of the second surface is angled relative to a plane perpendicular to the longitudinal axis. Optionally, the first surface and/or the second surface comprises a surface (79) that is parallel to the longitudinal axis.

Optionally, the sub-assembly comprises a feedback surface angled relative to a plane perpendicular to the longitudinal axis, wherein the feedback surface is spaced apart in a circumferential direction relative to the longitudinal axis from a proximal end of the first surface.

Optionally, the plunger rod comprises the feedback surface. Optionally, the feedback surface is parallel to the longitudinal axis.

Optionally, the component is rotationally locked relative to the housing (30, 62).

Optionally, a spring is arranged between the plunger rod and the housing, and the spring is configured to move the plunger rod towards the proximal end during medicament delivery.

Optionally, the plunger rod is rotationally locked relative to the housing by a protrusion and a groove arranged in the protrusion, wherein one of the protrusion and the groove is on the plunger rod and the other of the protrusion and the groove are on any component of the sub-assembly other than the plunger rod. Optionally, the any component of the sub-assembly other than the plunger rod is the housing.

Optionally, a distal end of the flexible arm is attached to a main body of the plunger rod, and wherein a proximal end of the flexible arm is free to flex relative to the main body of the plunger rod. Optionally, the flexible arm is in a recess or slot in a main body of the plunger rod.

An aspect concerns a medicament delivery device comprising any sub-assembly as described above. Optionally, the medicament delivery device is an autoinjector.

An aspect concerns a method of creating feedback in a medicament delivery device, the method comprising the following steps in the following order: moving a plunger rod in a proximal direction relative to another component so that a proximally facing surface of a flexible arm of the plunger rod engages a distally facing surface of the another component, thereby rotating the plunger rod in a first direction relative to the another component; moving the plunger rod further in the proximal direction relative to the another component so that the proximally facing surface of the plunger rod disengages the distally facing surface of the another component and subsequently rotates the plunger rod in a second direction, thereby creating feedback. Typically, the first direction and the second direction are opposite directions. Optionally, the another component is a housing.

An aspect concerns a sub-assembly for a medicament delivery device, the sub-assembly extending along a longitudinal axis in a longitudinal direction from a proximal end to a distal end, the sub-assembly comprising: a housing; and a plunger rod arranged at least partly inside the housing; wherein the plunger rod comprises a first engagement portion and a component of the sub-assembly other than the plunger rod comprises a second engagement portion, wherein the first engagement portion and the second engagement portion are arranged so that, when the plunger rod moves relative to the component during medicament delivery device use, the first engagement portion engages the second engagement portion so that a flexible arm of the plunger rod flexes relative to the component and then disengages the second engagement portion so that the flexible arm flexes back relative to the component. Optionally, the first engagement portion comprises a proximally facing surface. Optionally, the second engagement portion comprises a distally facing surface. Optionally, the first engagement portion and/or the second engagement portion is a protrusion.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a perspective view of a rear cap.

FIG. 2 shows a perspective view of part of a plunger rod.

FIG. 3 shows an example autoinjector that could comprise the sub-assemblies described herein.

FIGS. 4 and 5 show a perspective view of part of a plunger rod of an alternative example in which the only difference is that the flexible arm is a mirror image of the flexible arm shown in FIG. 2.

FIGS. 6 to 9 show alternative shapes for the protrusion of the plunger rod and for the flexible arm of the rear cap.

### DETAILED DESCRIPTION

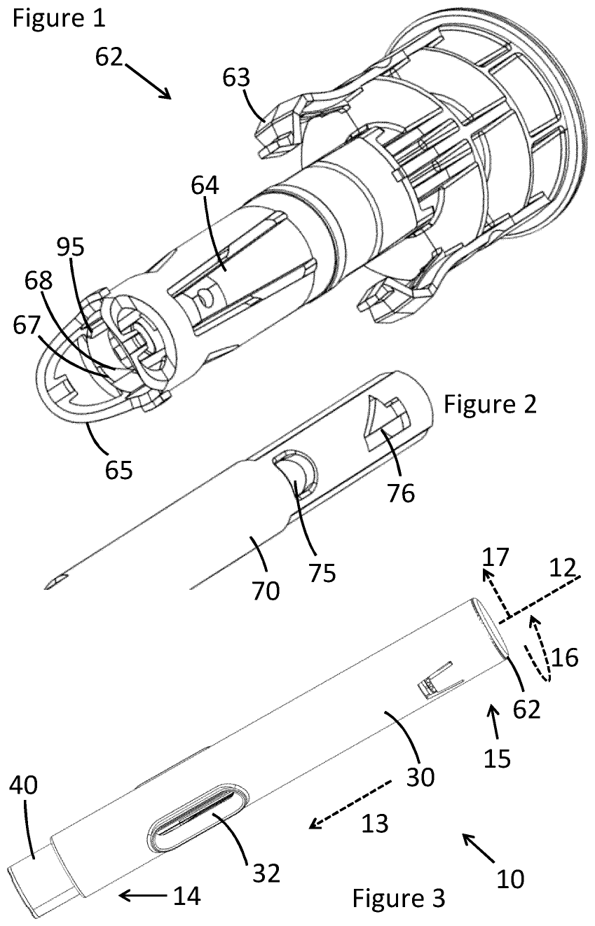

FIGS. 1 and 2 show the separate components of a sub-assembly for a medicament delivery device, namely a housing (in this case a rear cap 62) and a plunger rod 70.

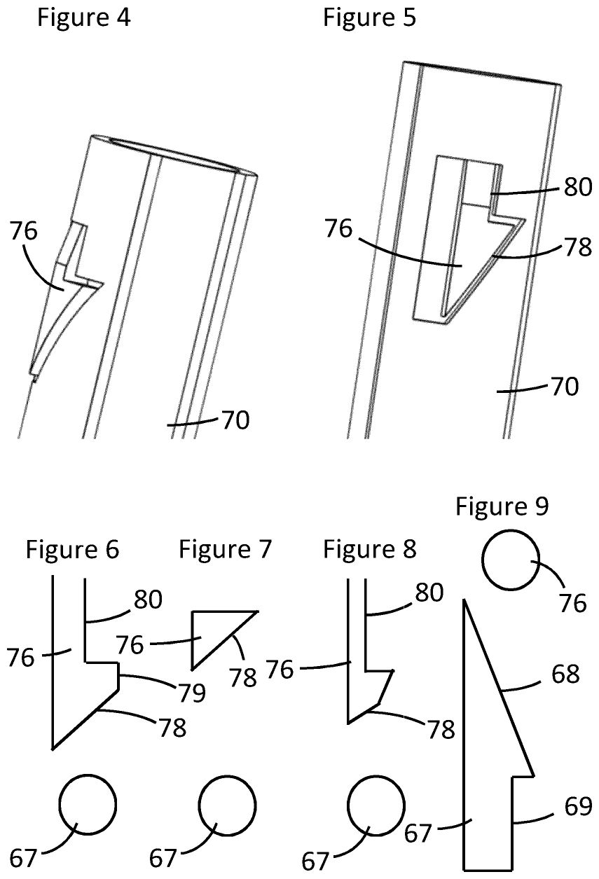

The plunger rod 70 comprises a proximally facing surface (first surface) facing towards the proximal end and a component of the sub-assembly other than the plunger rod (in this case the housing, and more particularly the rear cap 62) comprises a distally facing surface (second surface) facing towards the distal end. In the example shown in the figures, the proximally facing surface is a surface 78 of a flexible arm 76, where the flexible arm extends from the main body of the plunger rod 70 (see FIGS. 4 and 5 in particular for a similar example in which the only difference is that the flexible arm is a mirror image and therefore flexes in the opposite direction). As can be seen in FIG. 1, the distally facing surface is a surface 68 of a protrusion 67, where the protrusion extends from an inner surface of the rear cap 62. Optionally, as shown in FIG. 1, a second protrusion 95 can be provided on the rear cap; this can help prevent over-flexing of the flexible arm.

During use of a medicament delivery device comprising the sub-assembly, the plunger rod moves relative to the component (in this case the rear cap), resulting in the proximally facing surface and the distally facing surface engaging to flex the flexible arm relative to the component (and also relative to the main body of the plunger rod), and then subsequently disengaging so that the flexible arm flexes back relative to the component, which can create feedback (e.g. audible, visible and/or tactile feedback).

FIG. 3 shows an example of an autoinjector 10 which could comprise a sub-assembly as depicted in the other figures. For reference, a longitudinal axis 12, an axial direction 13 (longitudinal direction), a proximal end 14, a distal end 15, a circumferential direction 16 and a radial direction 17 are shown. The autoinjector comprises a housing comprising an outer housing 30 (with an optional window 32) and a rear cap 62, along with a needle guard 40 (more generally a medicament delivery member guard). A typical autoinjector would also include a powerpack to drive the plunger rod. The powerpack could provide power using a power source such as a spring, a battery or compressed gas. The rear cap 62 can be part of the powerpack, and could house the power source. A medicament container such as a syringe or a cartridge would be provided, and a medicament delivery member, such as a needle or a jet injector, would be provided. Although not essential, the powerpack would typically be locked until the device is activated for use, for example by a rotator being rotated by a medicament delivery member guard (e.g. needle guard 40), or by another component such as a button (see WO2011/123024, which is herein incorporated by reference, for an example of a rotator). A medicament delivery container carrier such as a syringe carrier could also be provided, although the medicament delivery container could be supported by one or more other components instead, such as by a housing. Although an autoinjector is described, the sub-assembly could also be used in other injectors or other medicament delivery devices more generally, including non-automatic medicament delivery devices such as pen injectors, and components such as the rear cap, the powerpack, the rotator and the medicament delivery member guard are therefore optional.

In general, during operation of the sub-assembly, the plunger rod moves in a proximal direction relative to another component so that a proximally facing surface of the plunger rod engages a distally facing surface of the another component, thereby flexing the flexible arm in a first direction relative to the another component. The plunger rod then moves further in the proximal direction relative to the another component so that the proximally facing surface of the flexible arm disengages the distally facing surface of the another component and subsequently the flexible arm flexes back in a second direction, thereby creating feedback. Typically, the first direction and the second direction are opposite directions. Typically, the flexing of the flexible arm is elastic.

This flexing back can be rapid, allowing for feedback (such as an audible click) due to an impact with another component.

The method of operation of the example sub-assembly in the figures will now be described in more detail. Once the plunger rod 70 has been released and is moving towards the proximal end of a medicament delivery device (i.e. moving in the proximal direction relative to the rear cap), the protrusion 67 of the rear cap and the flexible arm 76 of the plunger rod will engage one another. This can be at different points, for example this could be immediately upon the plunger rod moving (for feedback that an injection has started), at an intermediate point during injection, and/or at the end of an injection, for example.

Once the respective surfaces of the plunger rod and the rear cap engage one another, some of the energy from the power source that is driving the plunger rod is transferred to the flexible arm to flex the flexible arm.

Once the flexible arm has flexed to its full extent relative to the rear cap, continued movement of the plunger rod in the proximal direction relative to the rear cap will result in the respective surfaces of the plunger rod and the rear cap disengaging, which allows the energy built up in the flexible arm to be released, allowing the flexible arm to flex back.

The particular structure of the engagement portion of the plunger rod and the corresponding engagement portion of the rear cap will now be dealt with in more detail. In the example in the figures, a flexible arm is provided on the plunger rod, with a corresponding protrusion on the rear cap. The protrusion on the plunger rod could therefore be considered to be the engagement portion of the plunger rod, and the flexible arm of the rear cap could be considered to be the engagement portion of the rear cap.

As shown in more detail in the example in FIGS. 4 and 5, a flexible arm 76 is provided. The flexible arm comprises a surface 78 (rotation surface) and a surface 80 (impact surface). The surface 80 is spaced apart from the surface 78 (it is offset in the circumferential direction). This gap between the surface 78 and the surface 80 allows for acceleration of the flexible arm 76 relative to the protrusion 67, thereby allowing the impact of the protrusion 67 on the surface 80 to create feedback (in particular audible feedback). The surface 78 is a proximally facing surface, and is angled relative to the longitudinal axis-this angle allows the engagement between the protrusion 67 and the flexible arm 76 to rotate the plunger rod relative to the rear cap. In this example, the surfaces 78, 80 are adjacent to one another in the axial direction, but could be spaced apart, with an axially extending surface 79 (see FIG. 6 for an example) in between the surfaces 78, 80; this optionally axially extending surface would briefly hold the flexible arm flexed during an injection as the plunger rod continues to move in the proximal direction relative to the housing (rear cap in this example), and can be used to regulate the point during device use at which the energy built up in the torsion spring is released. The axially extending surface is optionally parallel to the longitudinal axis. The surface 80 is parallel to the longitudinal axis, though this is optional. The surface 80 is optional more generally as well, as a surface for the protrusion 67 to hit could be provided on another component. In addition, a surface for the protrusion 67 to hit is also optional, as the feedback could be tactile and/or visual instead of audible, for example with part or all of the flexible arm 76 visible through a window in an outer housing after the flexible arm 76 has passed the surface 78.

As shown in FIGS. 1 and 2, a flexible arm 76 with the shape shown in FIGS. 4 and 5 is provided (or more specifically as a mirror-image of the shape in FIGS. 4 and 5 in the example of FIGS. 1 and 2). In the examples, the flexible arm 76 is part of the plunger rod and is attached to the rest of the plunger rod (to the main body of the plunger rod) at one end of the flexible arm (the distal end of the flexible arm in this example, though it could alternatively be attached at the proximal end of the flexible arm), with the other end of the flexible arm free to flex relative to the rest of the plunger rod. The flexible arm 76 is partially within a recess or slot in the plunger rod; this is optional as the flexible arm could instead be arranged above the surface of the main body of the plunger rod, but it could be beneficial to have the flexible arm in a recess or slot to minimise the space requirements for the plunger rod. The protrusion comprises a surface 78 and a surface 80 as described above. Using a flexible arm means that energy can be stored in the arm by flexing the arm, meaning that energy to provide feedback can be stored in the flexible arm.

Providing more than one flexible arm 76 on the plunger rod could be beneficial. This plurality of flexible arms could be spaced out around the circumference of the plunger rod (with their corresponding protrusions correspondingly spaced out on another component). In such a case, each additional flexible arm (and corresponding protrusion) collects additional energy, thereby allowing for a stronger feedback (e.g. stronger audible feedback) due to the greater forces involved.

Although the examples herein provide the surfaces that engage one another as on a protrusion and on an outer surface of a flexible arm respectively, this is optional. For example, one of the surfaces could be on a protrusion, with the other surface being the wall of a slot (for example a helical slot), or one or more of the surfaces could be created by a discontinuity such as a step.

In the examples shown herein, the engagement portions (the surfaces that engage one another) are on the rear cap 62 and the plunger rod 70. Instead of being on the rear cap, the engagement portion could be on another component, such as on another part of the housing (e.g. an outer housing), a syringe carrier, or a rotator. A rotator in medicament delivery devices is typically rotatable relative to other components such as the housing and the plunger rod only at particular points during medicament delivery device use, and is rotationally restricted during other points, for example during medicament delivery. As such, the rotator can be used to house the engagement portion if the rotational state of the rotator is taken into account.

The plunger rod is typically rotationally locked relative to at least one other part of the sub-assembly, such as to the housing, for example by a protrusion and a groove arranged in the protrusion, wherein one of the protrusion and the groove is on the plunger rod and the other of the protrusion and the groove are on any component of the sub-assembly other than the plunger rod (e.g. on the housing, for example on the rear cap 62). The groove would typically be parallel to the longitudinal axis 12. Alternative mechanisms for restricting rotational movement of the plunger rod relative to one or more components could also be used; for example, when the powerpack comprises a compression spring as a power source, it may be that the spring can restrict rotational movement of the plunger rod relative to the housing by providing resistance to rotational movement of the plunger rod relative to the housing. In such a case, a protrusion and groove combination as outlined above would not be needed.

In the examples shown herein, the plunger rod 70 is held in place before use within the rear cap 62 by an arm 64 of the rear cap 62 interacting with a hole 75 of the plunger rod 70 (see FIGS. 1 and 2), although other mechanisms for holding the plunger rod in place prior to use could also be provided, for example a button-operated mechanism so that a user can activate the device by manually disengaging the plunger rod.

Various other modifications could be made to the example shown for the shape of the flexible arm 76 and the protrusion 67. A selection of alternative shapes for the flexible arm 76 and the protrusion 67 are shown in FIGS. 6 to 9. For example, the shape of the flexible arm of could be modified to include a shape as shown in one of FIGS. 6 to 9. A protrusion 67 of the housing (e.g. rear cap) is also shown for context. In FIGS. 6 to 8, the protrusion 67 of the housing is circular. In general, the protrusion 67 could be various shapes, including circular, triangular, square, or an irregular shape.

In the example in FIG. 1, the protrusion 67 of the rear cap has surfaces that are parallel to the longitudinal axis (the surface 68) and perpendicular to the longitudinal axis, and the only surface that is angled relative to the longitudinal axis is the surface 78 of the plunger rod. The two engagement portions could be switched, with the angled surface provided on the rear cap instead. An example where the angled surface is on the housing (e.g. rear cap), rather than on the plunger rod, is shown in FIG. 9. In this example, the surface 68 is angled in the same way as the surface 78 of the plunger rod in the examples in FIGS. 6 to 8, but with the surface 68 angled towards the distal end rather than towards the proximal end. The engagement portion of the flexible arm of the plunger rod in this example is circular, but could again be various other shapes, including triangular, square, or an irregular shape. A surface 69 (impact surface), which is analogous in function to the surface 80 of the other depicted examples but faces in the distal direction rather than in the proximal direction, is also optionally provided.

It is envisioned that two flexible arms 76 would be provided on the plunger rod, with two corresponding protrusion 67 on the rear cap. Typically, the two flexible arms 76 would be opposite each other relative to the longitudinal axis, and the two protrusions 67 would also be opposite to each other relative to the longitudinal axis, though this is optional and a different spacing could also be used. Alternatively, one, three or more flexible arms 76 could be provided, with the flexible arms optionally equally spaced around the axis. Generally, a protrusion 67 on the rear cap would be provided for each flexible arm 76 on the plunger rod.

Typically, at least one of the protrusion on the rear cap and the protrusion on the plunger rod comprises a surface (such as the surface 78) that is angled relative to a plane perpendicular to the longitudinal axis; that is, a surface that extends in a direction with an angle of greater than zero degrees and less than 90 degrees relative to a plane perpendicular to the longitudinal axis, for example between 10 and 85 degrees. Preferably, the angle is between 30 and 70 degrees, or between 45 and 60 degrees. The examples of the surface 78 shown in the figures all have a constant angle, but the angle could also vary, and could for example be curved when viewed from the angle shown in FIG. 5 (i.e. perpendicular to the longitudinal axis with the longitudinal axis behind the protrusion). As shown in FIG. 8, the surface 78 could also have two sections at different angles.

In the example in FIGS. 6, the flexible arm 76 comprises a surface 79 facing perpendicular to the axis in addition to the proximally facing surface 78. This surface 79 is optional, but can be useful to adjust the timing of feedback as mentioned above. A similar surface could be provided on the protrusion 67 shown in FIG. 9.

The particular shape of the rear cap 62 shown in the figures can be varied, and various other optional rear cap features, such as a powerpack transport locking arm 63, a pre-activation locking arm 64 and syringe support arms 65, are shown but are not described in detail as they are not directly relevant to providing feedback. The plunger rod is shown inside the rear cap in the depicted examples (i.e. the plunger rod is arranged at least partly inside the housing-at least partly inside the rear cap of the housing in these examples), with the protrusion of the rear cap extending from an inner surface of the rear cap, but this could be reversed, with the protrusion of the rear cap extending from an outer surface of the rear cap (for example from an outer surface of a tubular extension of the rear cap that extends inside the distal end of the plunger rod).

In this application, it is primarily envisioned that the feedback would be provided at the beginning or end of delivery, but the time of the feedback could be adjusted by adjusting the relative locations of the features described above. Optionally, multiple instances of feedback could be provided, for example with a selection from a start of delivery feedback, an end of delivery feedback, and ‘continuous’ feedback during delivery (i.e. multiple closely spaced clicks providing multiple instances of feedback). Two (or more) instances of feedback could be achieved by two (or more) flexible arms on the plunger rod at different axial positions and/or by two (or more) protrusions on another component (e.g. on a housing, such as the rear cap) at different axial positions. The feedback could be audible, tactile and/or visible, for example.

The drug delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

Some aspects are outlined in the following clauses.
1. A sub-assembly for a medicament delivery device (10), the sub-assembly extending along a longitudinal axis (12) in a longitudinal direction (13) from a proximal end to a distal end, the sub-assembly comprising:
   a housing (30, 62); and
   a plunger rod (70) arranged at least partly inside the housing (30, 62), the plunger rod (70) being rotationally locked relative to the housing (30, 62);
   wherein the plunger rod (70) comprises a flexible arm (76), the flexible arm (76) comprising a first surface (78) facing towards the proximal end; wherein a component of the sub-assembly other than the plunger rod (70) comprises a second surface (68) facing towards the distal end, with the first surface (78) and the second surface (68) being arranged so that, when the plunger rod (70) moves towards the proximal end relative to the component during medicament delivery device use, the first surface (78) and the second surface (68) engage to flex the flexible arm (76) in a circumferential direction (16) relative to the component and then disengage so that the flexible arm (76) flexes back relative to the component.
2. The sub-assembly of clause 1, wherein the component is the housing (30, 62).
3. The sub-assembly of clause 1 or 2, wherein the housing comprises an outer housing (30) and a rear cap (62), and the rear cap (62) comprises the second surface (68).
4. The sub-assembly of any previous clause, wherein at least part of at least one of the first surface (78) and the second surface (68) is angled relative to a plane perpendicular to the longitudinal axis (12).
5. The sub-assembly of clause 4, wherein the at least part of at least one of the first surface (78) and the second surface (68) is angled relative to a plane perpendicular to the longitudinal axis (12) at an angle of between 10 and 85 degrees.
6. The sub-assembly of clause 4 or 5, wherein at least part of the first surface (78) is angled relative to a plane perpendicular to the longitudinal axis (12).
7. The sub-assembly of clause 6, wherein the sub-assembly comprises a feedback surface (80) angled relative to a plane perpendicular to the longitudinal axis (12), wherein the feedback surface (80) is spaced apart in a circumferential direction (16) relative to the longitudinal axis (12) from a distal end of the first surface (78).
8. The sub-assembly of any of clauses 4 to 7, wherein at least part of the second surface (68) is angled relative to a plane perpendicular to the longitudinal axis (12).
9. The sub-assembly of clause 8, wherein the sub-assembly comprises a feedback surface (80) angled relative to a plane perpendicular to the longitudinal axis (12), wherein the feedback surface (80) is spaced apart in a circumferential direction (16) relative to the longitudinal axis (12) from a proximal end of the first surface (78).
10. The sub-assembly of clause 7 or 9, wherein the plunger rod (70) comprises the feedback surface (80).
11. The sub-assembly of any previous clause, wherein the component is rotationally locked relative to the housing (30, 62).
12. The sub-assembly of any previous clause, wherein a spring is arranged between the plunger rod and the housing, and the spring is configured to move the plunger rod towards the proximal end during medicament delivery.
13. The sub-assembly of any previous clause, wherein the plunger rod (70) is rotationally locked relative to the housing by a protrusion and a groove arranged in the protrusion, wherein one of the protrusion and the groove is on the plunger rod and the other of the protrusion and the groove are on any component of the sub-assembly other than the plunger rod.
14. The sub-assembly of clause 14, wherein the any component of the sub-assembly other than the plunger rod is the housing (30, 62).
15. The sub-assembly of any previous clause, wherein a distal end of the flexible arm is attached to a main body of the plunger rod, and wherein a proximal end of the flexible arm is free to flex relative to the main body of the plunger rod.
16. The sub-assembly of any previous clause, wherein the flexible arm is in a recess or slot in a main body of the plunger rod.
17. A medicament delivery device (10) comprising the sub-assembly of any previous clause.

1-15. (canceled)

16. A sub-assembly for a medicament delivery device, the sub-assembly extending along a longitudinal axis in a longitudinal direction from a proximal end to a distal end, the sub-assembly comprising:
a housing; and
a plunger rod arranged at least partly inside the housing, the plunger rod being rotationally locked relative to the housing,
wherein the plunger rod comprises a flexible arm, the flexible arm comprising a first surface facing towards the proximal end; wherein a component of the sub-assembly other than the plunger rod comprises a second surface facing towards the distal end, with the first surface and the second surface being arranged so that, when the plunger rod moves towards the proximal end relative to the component during medicament delivery device use, the first surface and the second surface engage to flex the flexible arm in a circumferential direction relative to the component and then disengage so that the flexible arm flexes back relative to the component.

17. The sub-assembly of claim 16, wherein the component is the housing.

18. The sub-assembly of claim 16, wherein the housing comprises an outer housing and a rear cap, and the rear cap comprises the second surface.

19. The sub-assembly of claim 16, wherein at least part of at least one of the first surface and the second surface is angled relative to a plane perpendicular to the longitudinal axis.

20. The sub-assembly of claim 19, wherein the at least part of at least one of the first surface and the second surface is angled relative to a plane perpendicular to the longitudinal axis at an angle of between 10 and 85 degrees.

21. The sub-assembly of claim 19, wherein at least part of the first surface is angled relative to a plane perpendicular to the longitudinal axis.

22. The sub-assembly of claim 21, wherein the sub-assembly comprises a feedback surface angled relative to a plane perpendicular to the longitudinal axis, wherein the feedback surface is spaced apart in a circumferential direction relative to the longitudinal axis from a distal end of the first surface.

23. The sub-assembly of claim 19, wherein at least part of the second surface is angled relative to a plane perpendicular to the longitudinal axis.

24. The sub-assembly of claim 23, wherein the sub-assembly comprises a feedback surface angled relative to a plane perpendicular to the longitudinal axis, wherein the feedback surface is spaced apart in a circumferential direction relative to the longitudinal axis from a proximal end of the first surface.

25. The sub-assembly of claim 16, wherein the component is rotationally locked relative to the housing.

26. The sub-assembly of claim 16, wherein the plunger rod is rotationally locked relative to the housing by a protrusion and a groove arranged in the protrusion, wherein one of the protrusion and the groove is on the plunger rod and the other of the protrusion and the groove are on any component of the sub-assembly other than the plunger rod.

27. The sub-assembly of claim 26, wherein the any component of the sub-assembly other than the plunger rod is the housing.

28. The sub-assembly of claim 16, wherein a distal end of the flexible arm is attached to a main body of the plunger rod, and wherein a proximal end of the flexible arm is free to flex relative to the main body of the plunger rod.

29. The sub-assembly of claim 16, wherein the flexible arm is in a recess or slot in a main body of the plunger rod.

30. A medicament delivery device comprising the sub-assembly of claim 16.
